(19) United States
(12) Patent Application Publication
(10) Pub. No.: US 2023/0357097 A1
(43) Pub. Date: Nov. 9, 2023

(54) A METHOD FOR THE TRANSFORMATION OF A LIQUID SUBSTRATE COMPRISING MICROORGANISMS, INTO A SUBSTANCE IN SOLID STATE AND RELATIVE SUBSTANCE

(71) Applicant: SYMBIAGRO SRL, RONCADELLE (BS) (IT)

(72) Inventors: Vittorio DE MICCO, TURATE (CO) (IT); Alfonso CERRATO, SARNO (SA) (IT)

(21) Appl. No.: 17/780,712

(22) PCT Filed: Nov. 18, 2020
(86) PCT No.: PCT/IT2020/050285

§ 371 Date: May 27, 2022

(30) Foreign Application Priority Data
Nov. 28, 2019 (IT) 102019000022365

(51) Int. Cl. C05F 11/08; A01N 63/20; A01N 63/30; A01N 25/12; C12N 1/20; C12N 1/14; C12M 1/06; C05G 5/18; C05G 5/30

(52) U.S. Cl. CPC C05F 11/08; A01N 63/20; A01N 63/30; A01N 25/12; C12N 1/20; C12N 1/14; C12M 27/02; C05G 5/18; C05G 5/30

(57) ABSTRACT

Disclosed is a method for the transformation of a liquid substrate including microorganisms, such as bacteria or fungi, into a substance in solid state, including: —production of a liquid substrate including a liquid culture of microorganisms through the use of a bioreactor; —providing a mixer provided with at least one nebulizer nozzle; —providing inside the mixer an adsorbent solid product such as amorphous silica; —nebulizing the liquid substrate including the liquid culture of microorganisms in the mixer through the at least one nebulizer nozzle; and mixing the liquid culture of microorganisms with the adsorbent solid product. Also disclosed is a substance in solid state including an amorphous substrate and microorganisms adhering to the substrate, obtained using a method as described above.

### CROSS-REFERENCE TO RELATED APPLICATIONS

This application is the U.S. national phase of International Application No. PCT/IT2020/050285 filed Nov. 18, 2020 which 10 designated the U.S. and claims priority to IT Patent Application No. 102019000022365 filed Nov. 28, 2019, the entire contents of each of which are hereby incorporated by reference.

### BACKGROUND OF THE INVENTION

### Field of the Invention

The invention relates to the biological sector of methods for the transformation of a culture of microorganisms in liquid state into a solid state.

More specifically, the invention relates to a method for the transformation of a liquid substrate comprising microorganisms, such as bacteria or fungi, into a substance in solid state, while preserving the viability of the strains for a long time, particularly for use in agriculture.

The invention also relates to the substance in solid state directly obtained by this transformation method.

### Description of the Related Art

The most commonly used current techniques for transforming a substrate from liquid to solid are:

Freeze-drying: this is a drying technique that, starting from a solution, makes it possible to obtain a porous, friable, hygroscopic solid having a large specific surface area and being rapidly soluble (freeze-dried). The product is obtained by freezing the solution followed by vacuum sublimation of the solvent. The steps that characterise freeze-drying are:
freezing;
vacuum sublimation (primary drying);
desorption (secondary drying).

This technique involves the use of complex machinery dedicated exclusively to freeze-drying.

The freeze-drying technique makes it possible to obtain:
a solid that is stable over time, starting from easily degradable substances;
immediate solubilization of the solid in the solvent;
a sterile solid to reconstitute.

The advantages of this technique are:
little loss of activity for delicate, thermolabile products;
obtaining a porous, immediately rehydratable product;
the possibility of obtaining sterile products;
precise, accurate dosage of product (liquid) into the final containers.

However, this technique also has certain drawbacks, such as the high cost of machinery, high energy costs and very long processing times (on average 24 hours/cycle).

There also exist certain specific problems linked to freeze-drying for use in the agricultural sector: the method is very costly and therefore not easy to use in all contexts, requiring process optimisation, specific skills and numerous experiments. Furthermore, when dealing with microorganisms, final viability may be considerably reduced by the presence of extreme conditions such as cold and high vacuum conditions.

Spray-drying: this is a technique involving the drying of a fluid current which is reduced to solid particulate by sub-dividing it into fine drops and striking it with a current of hot air (or another gas). Based on the type of fluid dried (solution, suspension, paste, etc.), the solid product may be a fine powder, granules or agglomerates. The drying process involves four steps:
atomization of the current;
air-spray contact;
drying of the spray;
separation of the dry product from the air.

Actuation of this method involves the use of very complex, and once again dedicated machinery.

Spray drying has several advantages:
it can be used for thermolabile substance, thanks to the short contact time;
it makes it possible to condition the dimension and final level of humidity of the particles;
it makes it possible to obtain a constant powder quality;
it is a continuous, single-step operation;
it guarantees flexibility at the design stage.

On the other hand, disadvantages associated with this method include: a high initial cost of investment, low thermal efficiency, little flexibility during “work in progress” and low product density without further processing.

Both techniques described, and currently most widely used, also have several disadvantages in common, especially when used for the manufacture of products for the agricultural industry.

First of all, the high costs involved, since both techniques require very costly machinery both in terms of start-up (purchase) and in terms of running and use (maintenance, utility costs, high energy consumption). Furthermore, the machinery is very bulky, requiring dedicated company spaces to be set aside.

Another aspect to consider is the exclusiveness of the machinery currently available. When buying a freeze-drier or a spray-drier, the instrument can only be used for that specific purpose.

Both freeze-drying, which requires a highly complex initial design stage to optimise the finished product, and spray-drying, which does not allow the product to be modulated during production, are complex, rigid techniques.

CN 105 272 459 A discloses a procedure for obtaining a composite fertilizer, comprising at least two microorganism-based products that are mixed and then re-suspended in a soil for growth. The procedure creates a composite fertilising product, containing a plurality of microorganisms, in a liquid formulation.

US 2013/236522 A1 discloses a procedure that comprises the growth of microorganisms in a liquid substrate and a subsequent drying phase using known freeze-drying or spray-drying processes.

E. Malusà et Al. “Technologies for Beneficial Microorganism Inocula Used as Biofertilizer” THE SCIENTIFIC WORLD JOURNAL, vol. 133, no. 3, 1 Jan. 2012 (2012 Jan. 2001), pages 3473-12, XP055325563, DOI: 10.1100/2012/491206 discloses various examples of procedures for the production of both solid and liquid microorganism-based inoculants, using known laboratory techniques, that enable the microorganisms to survive for a short time when stored at low temperatures, making them unsuitable for commercial use, which requires considerably longer life times.

U.S. Pat. No. 4,956,295 A discloses a procedure for obtaining a stable solid product, but the microorganisms are subjected to a traditional high-temperature drying stage, with consequent high process costs.

US 2008/107689 A1 discloses a procedure for growing and developing a microorganism on a solid substrate until complete colonisation. The resultant product is a humid paste that can be stored at low temperatures and used as a fertiliser only after being dissolved in water.

U.S. Pat. No. 9,090,884 B2 discloses a procedure for obtaining a product containing a formulation comprising a plurality of vital organisms and micro-encapsulating agents. The process is complex for the number of phases and components involved, and the product is consequently very costly.

US 2019/194600 A1 discloses a procedure for producing a porous substance on which to multiply, or grow, microorganisms. Growth of the microorganisms is difficult to control, however, and the duration and methods of conservation of the product are not defined.

WO 00/29544 A1 discloses a method for extracting active compounds deriving from a fermentation process conducted in a specific bioreactor. It therefore does not involve the direct use of the microorganisms developed in the reactor.

### SUMMARY OF THE INVENTION

The invention proposes to overcome these limits and defects.

The main object of the invention is to define a method for the transformation of a liquid substrate comprising microorganisms, into a substance in solid state, of a powdery type, while preserving the viability of the strains of microorganisms, such as bacteria or fungi.

A further object of the invention is to define a process that is efficient, flexible, modulable, and can be implemented over time, involving machinery and instrumentation that require a minimum investment cost, occupy little space, can also be used for other activities and enable the process to be modulated.

Another object is to use the transformation method to obtain a substance in solid state that is economical, can be stored for a long time at ambient temperature, with the viability of the microorganisms stable over time, that does not aggregate, is extremely versatile and suitable for use in agriculture in various agronomic applications, from fertigation to more traditional methods, mixed with other solid or liquid products, or on its own in formulations with a high content of active microorganisms.

These objects are achieved with a method for the transformation of a liquid substrate into a substance in solid state as disclosed and claimed.

In particular, the objects are achieved with a method that makes a liquid culture become solid by mixing it with an inert compound, such as synthetic amorphous silica for example, so as to obtain a constant humidity (even very low) while preserving the viability of the microorganisms for a long time.

A further object of the invention is a substance in solid state, particularly a powder, directly obtained through this process.

The method makes it possible to obtain the following main advantages:
the viability of the strains is preserved without the need for sporulation induction prior to the treatment;
the technique is particularly suitable for microorganisms capable of producing forms of resistance (spores);
costs are reduced: most of the machinery used in the process are potentially already present on the factory premises, without the need for further purchases;
the process is performed at ordinary temperatures: there is no need to provide sources of cooling or heating, the process takes places at ambient temperature, with considerable savings in terms of energy and the environment;
the scientific instruments used can also be used for other activities;
the system can be modulated while in progress, and since it is an open system, it is possible to obtain product concentrations and humidity levels that can be modulated based on needs;
the viability of the microorganisms is preserved during the passage from the liquid phase to the solid product phase;
conservation over time of the product obtained is guaranteed, on a par with or superior to standard methods;
the process can be conducted in non-sterile conditions: once a starting product is obtained containing only the microorganism of interest, the process allows for processing in non-sterile conditions. In fact, by mixing the liquid culture with an amorphous silica-based solid product, possible contaminations during the process are reduced by the decrease in residual humidity. It will therefore be possible to obtain a product in which only the resistant microorganisms introduced survive, while any contaminations will not be able to replicate, thus remaining at minimal levels;
the percentage of solid product to be added can be easily changed based on the type of product one wants to obtain, with a higher or lower degree of humidity, different degrees of smoothness or different final concentrations.

### BRIEF DESCRIPTION OF THE DRAWINGS

The advantages of the invention will become more apparent from the following preferred mode of implementation of the invention, given by way of non-limiting illustration, and with the help of the figures, where:

FIG. 1 shows a schematic depiction of the steps involved in the method for the transformation of a liquid substrate into a substance in solid state;

FIG. 2 shows a schematic depiction of the substance in solid state directly obtained with this transformation method.

### DESCRIPTION OF THE PREFERRED EMBODIMENTS

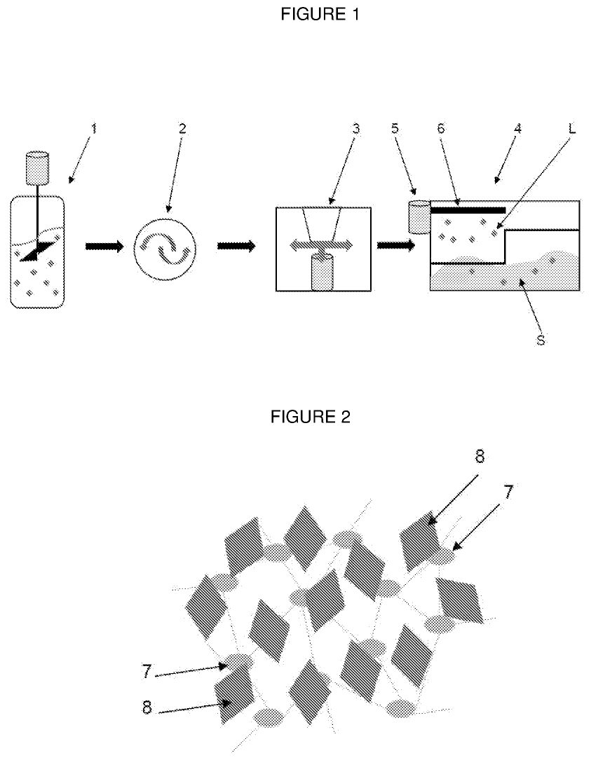

With reference to FIG. 1, the process object of the present inventions, through which it is possible to bring a liquid culture or microorganisms to a solid state, comprises the following steps:
production of the liquid culture of microorganisms through the use of a bioreactor 1 or classical fermentation methods (such as flasks and shakers);
centrifuging by means of a centrifuge 2 for concentration of the microorganisms, if needed;
micronisation by means of a microniser 3, this step being necessary to make the liquid produced homogeneous in the case of bacterial growths rich in frustules or fungals with the presence of solid mass;
mixing, by means of a mixer 4, advantageously of a double ribbon type, provided with at least one nebulizer nozzle 6 through which it is possible to introduce by spray the liquid substrate, so as to mix the liquid culture of microorganisms with the adsorbent solid product.

More specifically, the first step involves the production of a liquid culture through a process of fermentation. This step can be carried out using a bioreactor 1 or in laboratory conditions.

Excellent results have been obtained using 50 and/or 500-litre bioreactors.

Once fermentation has taken place, the liquid product is drained from the bioreactor 1 and if necessary passed through the centrifuge 2 (max. speed 9600 rpm), so as to remove excess liquid and thereby concentrate the product. This step can be avoided if considered unnecessary, for example in the case of high titre concentrations or particular production needs. This step is also modulable, meaning that it is possible to obtain a more or less liquid product.

The liquid product—as is or centrifuged—is used for micronisation, a step in which a pump pushes the liquid through a turbine, which micronises any solid components present in the product (frustules, fungal myceleum), producing a homogeneous product.

The processed product is then sent to the final phase, mixing. Through a pump 5, the liquid L is aspirated and pushed through nozzles 6 (with variable dimensions depending on needs) that nebulise the product on the adsorbent solid S, advantageously amorphous silica in powder form.

In this phase, it is possible to modulate the dimensions of the drops of liquid L and the quantity of solid S, so that it will be possible to obtain a highly-flexible product based on needs.

Once this phase is completed, the product is drained from the mixer and can be stored in bulk or bagged directly, based on needs.

With reference to FIG. 2, a schematic depiction is shown of the substance in solid state directly obtained using the transformation method described above, where the elliptical structures 7 represent the amorphous substrate and the rhomboid structures 8 represent the microorganisms adhering to the substrate.

The solid substrate used (amorphous silica) is in powder form, using the mixer jet one obtains a solid substrate also in powder form, to which the microorganisms can adhere.

The substance in solid state obtained with this process is a fine powder, with variable granule size, 40% of which can pass through a 60 mesh (250 micron) sieve without the need for further processing. The humidity of the substance is on average between 20% and 60%, but processing can also be conducted with variable humidity, based on the characteristics of the final product one wishes to obtain.

The powder containing microorganisms can be added as an additive to other formulations used in the agricultural sector, whether liquid or solid, and is therefore highly versatile. Otherwise the product can be used as the sole component of formulations with a high technological value, i.e. without chemical additives, substantially containing only pure microorganisms, capable of performing an active function on the soil without compromising the viability of the other microorganisms already present.

### Example 1

40 L of soil suitable for the development of the bacterial microorganism under study were inserted in a bioreactor 1. Inoculation was carried out and the growing conditions needed for multiplication of the bacterium were applied.

At the end of the fermentation process the product was drained from the bioreactor and stored until the mixing phase.

The liquid product was fed to the mixer 4 and nebulized, using small-dimension nozzles 6, directly into the device containing only the adsorbent solid product S (amorphous silica).

Mixing of the two components was performed for several hours, in order to obtain a uniform product. The product was then drained and stored in suitable containers until required for use.

The result of this process was a fine powder, with a residual humidity between 20% and 60%.

The viability of the product thus obtained was at least one year, with storage at ambient temperature.

### Example 2

30 L of soil suitable for the development of the fungal microorganism under study were inserted in a bioreactor 1. Inoculation was carried out and the growing conditions needed for multiplication of the fungus were applied.

At the end of the fermentation process the product was drained from the bioreactor 1 and stored until the micronisation phase.

The liquid fungal product contained a significant solid proportion, mycelium, requiring passage through a microniser 3.

Following a series of cycles in the microniser 3 the product had suitable dimensions, and it was thus possible to move on to the next phase.

The micronised product was fed to the mixer 4 and nebulized, using medium-dimension nozzles 6, directly into the device containing only the adsorbent solid product S (amorphous silica).

Mixing of the two components was performed for several hours, in order to obtain a uniform product. The product was then drained and stored in suitable containers until required for use.

The result of this process was a fine powder, with a residual humidity between 20% and 60%.

The viability of the product thus obtained was at least one year, with storage at ambient temperature.

1. A method for the transformation of a liquid substrate comprising microorganisms into a substance in solid state, comprising:
producing a liquid substrate comprising a liquid culture of microorganisms through the use of a bioreactor;
providing a mixer provided with at least one nebulizer nozzle;
providing inside the mixer an adsorbent solid product;
nebulizing the liquid substrate comprising the liquid culture of microorganisms in said mixer through said at least one nebulizer nozzle;
mixing the liquid culture of microorganisms with the adsorbent solid product.

2. The method according to claim 1, wherein said step of providing a mixer comprises the step of providing a mixer of a double ribbon type.

3. The method according to claim 1, further comprising the step of centrifuging said liquid culture of microorganisms prior to introduction into said mixer.

4. The method according to claim 1, further comprising the step of micronize said liquid culture of microorganisms prior to introduction into said mixer.

5. The method according to claim 1, further comprising the step of modulating the dimensions of the drops and the quantity of solid to obtain a substance in solid state with a humidity value between 20% and 60%.

6. Substance in solid state comprising an amorphous substrate and microorganisms adhering to said substrate, wherein the substance is obtained using the method according to claim 1.

7. The substance according to claim 6, wherein said amorphous substrate comprises amorphous silica.

8. The substance according to claim 6, wherein said microorganisms comprise bacteria or fungi.

9. The substance according to claim 6, wherein the substance is in the form of a fine powder.

10. An additive to other formulations used in the field of agriculture comprising the substance according to claim 6.

11. Substance in solid state comprising an amorphous substrate and microorganisms adhering to said substrate, wherein the substance is obtained using the method according to claim 2.

12. Substance in solid state comprising an amorphous substrate and microorganisms adhering to said substrate, wherein the substance is obtained using the method according to claim 3.

13. Substance in solid state comprising an amorphous substrate and microorganisms adhering to said substrate, wherein the substance is obtained using the method according to claim 4.

14. Substance in solid state comprising an amorphous substrate and microorganisms adhering to said substrate, wherein the substance is obtained using the method according to claim 5.

15. An additive to other formulations used in the field of agriculture comprising the substance according to claim 7.

16. An additive to other formulations used in the field of agriculture comprising the substance according to claim 11.

17. An additive to other formulations used in the field of agriculture comprising the substance according to claim 12.

18. An additive to other formulations used in the field of agriculture comprising the substance according to claim 13.

19. An additive to other formulations used in the field of agriculture comprising the substance according to claim 14.

20. The method of claim 1, wherein the adsorbent solid product is amorphous silica.
